(19) United States
(12) Patent Application Publication
(10) Pub. No.: US 2026/0217647 A1
(43) Pub. Date: Jul. 30, 2026

(54) CANCER CELL GROWTH INHIBITING COMPOSITION AND PROCESSED FOOD

(71) Applicants: HAGIHARA FARM PRODUCTION INSTITUTE CO., LTD., Nara (JP); KINKI UNIVERSITY, Osaka (JP)

(72) Inventors: Takashi KITAYAMA, Kyoto (JP); Gengo KASHIWAZAKI, Nara (JP); Toshiharu HASHIZUME, Nara (JP)

(73) Assignees: HAGIHARA FARM PRODUCTION INSTITUTE CO., LTD., Nara (JP); KINKI UNIVERSITY, Osaka (JP)

(21) Appl. No.: 19/148,403

(22) PCT Filed: Jan. 19, 2024
(86) PCT No.: PCT/JP2024/001503

§ 371 Date: Jul. 16, 2025

(30) Foreign Application Priority Data
Jan. 27, 2023 (JP) 2023-010871

(51) Int. Cl. C07C 381/12; A61K 31/10; A61P 35/00; C07C 317/08

(52) U.S. Cl. CPC C07C 381/12; A61K 31/10; A61P 35/00; C07C 317/08

(57) ABSTRACT

It is known that phytol contained in a watermelon sprout has a cancer cell growth-inhibiting effect. However, a major limitation is that a relatively high dose is required to achieve this effect. A cancer cell growth-inhibiting composition comprising, as an active ingredient, at least one compound having a structure represented by formula (123), or a pharmaceutically acceptable salt thereof, exhibit a stronger inhibitory effect on cancer cell proliferation and a lower inhibitory effect on normal cell growth compared to phytol. Therefore, it may serve as an anticancer agent with excellent selectivity toward cancer cells.

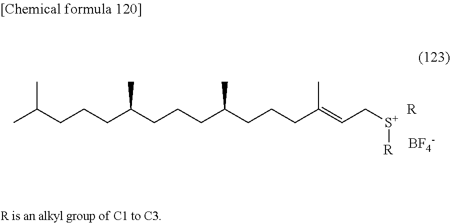

### TECHNICAL FIELD

The present invention relates to a pharmaceutical composition and a processed food comprising a sulfur-containing phytol-based compound, which exhibit an inhibitory effect on cancer cell proliferation.

### BACKGROUND ART

It is known that a watermelon sprout extract exhibits an inhibitory effect on cancer cell growth (Patent literature 1). This literature describes that phytol and lutein in the watermelon sprout extract are particularly effective in exerting this effect. Furthermore, since these substances do not adversely affect normal cells, they are expected to serve as active ingredients in anticancer pharmaceutical compositions with reduced side effects.

### CITATION LIST

### Patent Literature

PTL 1: International Publication No. 2017/131175

### SUMMARY OF INVENTION

### Technical Problem

Phytol and lutein have been expected to be used in an anticancer pharmaceutical compositions. However, one of the challenges has been the requirement of high dosages in order to achieve efficacy.

### Solution to Problem

The present invention has been conceived in view of the above-described problem and aims to provide a substance having a high cancer cell growth-inhibiting effect.

More specifically, a cancer cell growth-inhibiting composition according to the present invention is characterized in that it comprises, as an active ingredient, at least one compound having a structure represented by formula (123), or a pharmaceutically acceptable salt thereof.

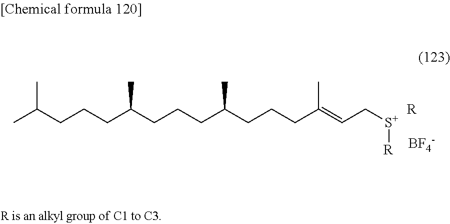

Note that the present invention can also be provided in the form of a processed food. More specifically, a processed food according to the present invention is characterized in that it comprises, as an active ingredient, at least one compound having a structure represented by formula (123) or a pharmaceutically acceptable salt thereof. Further, formula (123) encompasses formulae (1), (2), and (3). In addition, a compound having a structure represented formula (4) or formula (5) has been synthesized for the first time, and is therefore considered a novel compound.

Note that the characters “PT42,” “PT43,” “PT44,” “PT40,” and “PT41” in formulae (1) to (5) are provisional identifiers for the respective compounds described herein, and are not part of the chemical structures themselves.

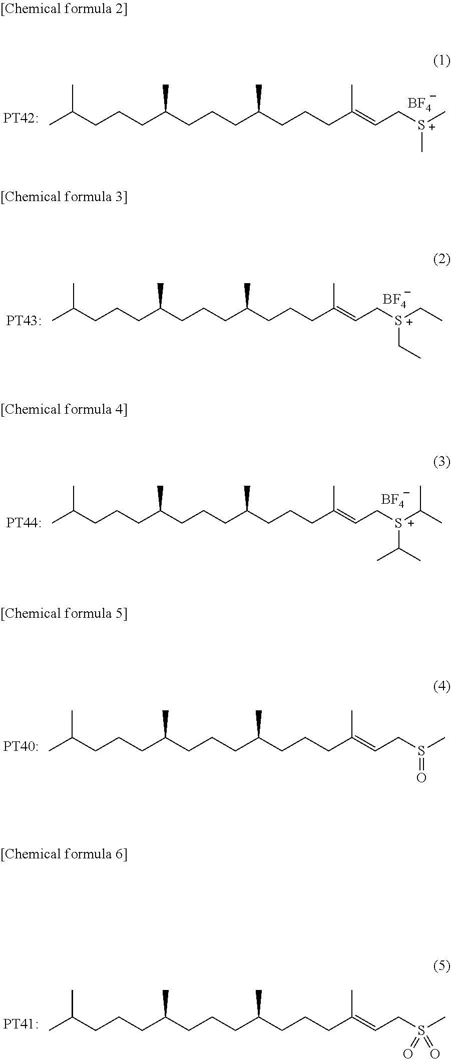

### Advantageous Effects of Invention

The cancer cell growth-inhibiting composition according to the present invention is capable of inhibiting the proliferation of cancer cells. Therefore, it can be suitably used as an anticancer pharmaceutical composition. In addition, the processed food according to the present invention comprise a compound exhibiting a cancer cell growth-inhibiting effect, so that cancer may be prevented by regularly ingesting the processed food in the form of a supplement or the like.

### DESCRIPTION OF EMBODIMENTS

The cancer cell growth-inhibiting composition and the processed food according to the present invention will be described below with reference to examples. It should be noted that the following description illustrates one embodiment and one example of the present invention, and the present invention is not limited thereto. The description may be modified without departing from the scope and spirit of the present invention.

The cancer cell growth-inhibiting composition according to the present invention comprises a compound having a structure represented by any one of formulas (1) to (5), or a pharmaceutically acceptable salt thereof.

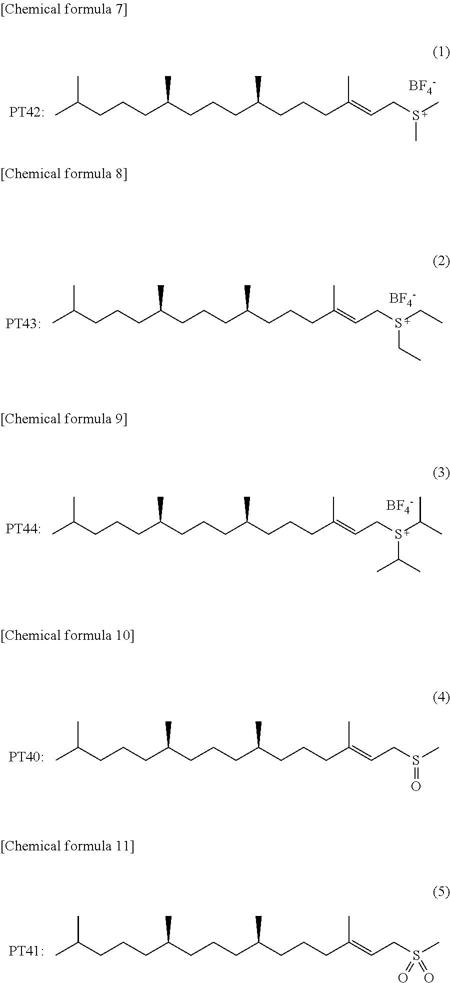

The compound of the formula (1) is referred to as “PT42,” the compound of the formula (2) is referred to as “PT43,” the compound of the formula (3) is referred to as “PT44,” the compound of the formula (4) is referred to as “PT40,” and the compound of the formula (5) is referred to as “PT41.” Furthermore, the formulas (1), (2), and (3) are collectively represented as the formula (123).

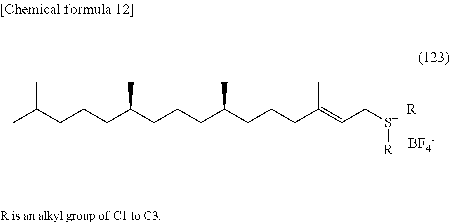

Note that R as used herein is an alkyl group of C1 to C3.

When these compounds are used as a cancer cell growth-inhibiting composition (pharmaceutical composition), they may be used alone or as salts obtained by mixing the compounds with a pharmaceutically acceptable acid in a solvent such as, for example, water, methanol, ethanol, or acetone. Examples of pharmaceutically acceptable acids include inorganic acids such as hydrochloric acid, hydrobromic acid, sulfuric acid salt, phosphoric acid, and nitric acid, and organic acids such as acetic acid, propionic acid, oxalic acid, succinic acid, lactic acid, malic acid, tartaric acid, citric acid, maleic acid, fumaric acid, methanesulfonic acid, p-toluenesulfonic acid, and ascorbic acid.

The mode of administration of the cancer cell growth-inhibiting composition of the present invention, when used as a medical composition, is not particularly limited, and may be either oral or parenteral. Further, depending on the administration route, the aforementioned composition can be prepared as an appropriate dosage form, for example, as injectable preparations, oral preparations such as capsules, tablets, granules, powders, pills, and fine granules, or various other preparations such as rectal preparations, lipophilic suppositories, and hydrophilic suppositories.

When the cancer cell growth-inhibiting composition of the present invention is used as a pharmaceutical composition, the proportional of the composition as an active ingredient may be adjusted according to the conditions of the use. That is, the administration concentration may be increased to a level sufficient to inhibit the proliferation of the target cancer cells.

The cancer cell growth-inhibiting composition according to the present invention may also be provided in the form of a processed food. Examples of such processed foods include general processed foods, discretionary foods, and health foods such as candy, gum, jelly, biscuits, cookies, rice crackers, bread, noodles, fish and meat paste products, tea, soft drinks, coffee drinks, milk beverages, whey drinks, lactic acid bacteria beverages, yogurt, ice cream, and pudding; foods with health claims such as foods for specified health uses and foods with nutrient function claims, as defined under the “Food with Health Claims system” of the Ministry of Health Labor and Welfare of Japan; nutritional supplements, animal feed, and food additives.

The processed food according to the present invention can be prepared by adding the cancer cell growth-inhibiting composition to the raw materials of these processed foods.

### EXAMPLES

### 1. Synthesis of Compound

First, as a starting material, (E)-1-bromo-3, 7, 11, 15-tetramethylhexadec-2-ene: (E)-1-bromo-3, 7, 11, 15-tetramethylhexadeca-2-ene represented by the formula (C3) was synthesized.

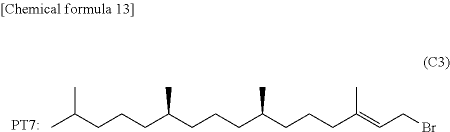

To a 200-mL two-necked flask was added phytol (CAS No. 7541-49-3) (500 mg, 1.69 mmol) dissolved in dry diethyl ether (Et₂O, 9 mL), and the solution was stirred at 0° C. for a short period. Phosphorus tribromide (0.057 mL, 0.59 mmol, 0.4 equiv) was then added dropwise at the same temperature and the mixture stirred for additional 30 min. It should be noted that phytol possesses a structure represented by formula (C1).

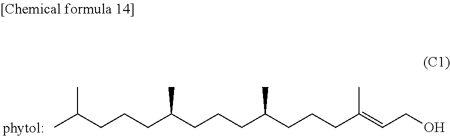 [SMILES: C/C(=C\CO)CCC[C@H](C)CCC[C@H](C)CCCC(C)C ; "phytol:"]

The reaction progress was monitored by TLC (Hexane (hexane)/AcOEt (ethyl acetate)=10/1). After confirming the disappearance of the starting material on TLC, the reaction was quenched by dropwise addition of saturated aqueous sodium bicarbonate. The reaction mixture was extracted with ethyl acetate, and the organic layer was collected, neutralized with saturated aqueous sodium bicarbonate, washed with brine, and dried over anhydrous sodium sulfate. The drying agent was removed by gravity filtration, and the filtrate was concentrated under reduced pressure using a rotary evaporator to afford a crude red-brown oily product (660.8 mg). No further purification was performed.

The NMR spectrum of the product obtained as described above is shown below.

(reddish brown oil) ¹H NMR (400 MHz, CDCl₃): δ=0.82-0.89 (m, 14H), 1.02-1.41 (m, 21H), 1.50-1.62 (m, 2H), 1.72 (d, 3H, J=1.3 Hz), 2.02 (t, 2H, J=7.6 Hz), 4.04 (d, 2H, J=8.4 Hz), 5.53 (t, 1H, J=8.4 Hz)

Based on the above results, the product was determined to have the structure shown in formula (C3). Hereinafter, the compound represented by formula (C3) is referred to as “PT7.”

<1-1>
Synthesis of Formula (C2) (referred to as “PT37”)

To a solution of sodium thiomethoxide (258 mg, 3.7 mol, 2.0 equiv) in methylene chloride solution (7 mL) was added a solution of PT7 (0.66 g, 1.8 mmol, 1.0 eq) in methylene chloride (7 mL) at −20° C. After stirring at the same temperature for 2 h, the temperature was gradually warmed to room temperature over 16 h. Reaction completion was confirmed by TLC (developed with hexane). Water was added, and the mixture was extracted three times with methylene chloride. The combined organic layers were dried over sodium sulfate, and then the solvent was removed under reduced pressure. Purification by Silica gel column chromatography (hexane/ethyl acetate=80/1) afforded PT37 as a colorless oil (346 mg, 63%).

The NMR Chemical shifts and the mass spectrometric data for the product obtained as described above are presented below.

¹H NMR (CDCl₃, 400 MHz): δ 5.23 (1H, tq, J=7.9, 1.2 Hz, CH at C2), 3.13 (2H, d, J=7.8 Hz, CH₂ at C1), 2.03 (3H, s, —SCH3), 2.01 (2H, t, J=7.6 Hz, CH₂ at C4), 1.64 (3H, d, J=0.7 Hz, CH₃ at C3), 1.57-1.47 (3H, sept, J=6.6 Hz, CH at C15), 1.46-0.99 (21H, m), 0.87 (6H, d, J=6.7 Hz, CH₃ at C15), 0.85 (3H, d, J=6.6 Hz, CH₃ at C11 or C7), 0.84 (3H, d, J=6.6 Hz, CH₃ at C11 or C7). 13C NMR (CDCl₃, 100 MHz): δ 139.4, 120.0, 39.9, 39.4, 37.5, 37.4, 37.4, 37.3, 36.6, 32.8, 32.7, 31.2, 28.0, 25.3, 24.8, 24.5, 22.7, 22.6, 19.8, 16.0, 14.5.

Based on these analytical results, the product confirmed to be the compound shown in formula (C2).

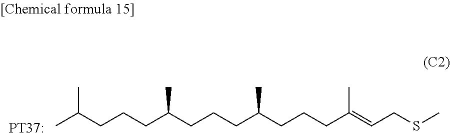

<1-2>

### Synthesis of Formula (4) (“PT40”)

To a solution of PT37 (formula (C2)) (170 mg, 0.52 mmol, 1.0 equiv) in acetonitrile (2.6 mL) was added an aqueous solution (0.27 mL) of sodium hypochlorite pentahydrate (250 mg, 1.5 mmol, 2.2 equiv). The reaction mixture was stirred for 20 min, and the reaction progress was monitored by TLC (chloroform/methanol=19:1). Upon completion, aqueous sodium sulfite was added, and the mixture was extracted three times with ethyl acetate. The combined organic layers were dried over sodium sulfate, and the solvent was removed under reduced pressure. Purification by silica gel column chromatography (chloroform) afforded PT40 as a colorless oil (38 mg, 16%).

The NMR chemical shifts and mass spectrometric data for the product obtained in this procedure are shown below.

¹H NMR (CDCl₃, 400 MHz): δ 5.24 (1H, tq, J=8.0, 1.1 Hz, CH at C2), 3.58 (1H, dd, J=13.0, 8.0 Hz, CH₂ at C1), 3.43 (1H, dd, J=13.0, 8.0 Hz, CH₂ at C1), 2.53 (3H, s, —SCH3), 2.06 (2H, t, J=7.3 Hz, CH₂ at C4), 1.72 (3H, s, CH₃ at C3), 1.52 (1H, sept, J=6.6 Hz, CH at C15), 1.46-0.99 (25H, m), 0.87 (6H, d, J=6.6 Hz, CH₃ at C15), 0.85 (3H, d, J=6.6 Hz, CH₃ at C11 or C7), 0.84 (3H, d, J=6.6 Hz, CH₃ at C11 or C7). 13C NMR (CDCl₃, 100 MHz): δ 145.9, 110.7, 53.6, 40.1, 39.4, 37.4, 37.4, 37.3, 37.3, 36.6, 32.8, 32.7, 28.0, 25.2, 24.8, 24.4, 22.7, 22.6, 19.8, 19.7, 16.9. HRMS m/z [M+Na]⁺ Calcd for C₂₁H₄₂OS 365.2854; Found 365.2858.

<1-3>

### Synthesis of PT41 (Formula (5))

To a solution of PT37 (formula (C2)) (480 mg, 1.5 mmol, 1.0 equiv) in toluene (4.4 mL) was added an aqueous solution (1.3 mL) of sodium hypochlorite pentahydrate (580 mg, 3.5 mmol, 2.4 equiv), and the mixture was stirred for 3 h. The progress of the reaction was monitored by TLC (hexane/ethyl acetate=5:1). Upon completion of the reaction, confirmed by TLC, aqueous sodium sulfite was added, and the mixture was extracted three times with ethyl acetate. The combined organic layers were dried over sodium sulfate, and the solvent was removed reduced pressure. Purification by Silica gel column chromatography (hexane/ethyl acetate=50:1) afforded PT41 as a colorless oil (45 mg, 9%).

The NMR chemical shifts and mass spectrometric data for the product of the product obtained as described above are presented below.

¹H NMR (CDCl₃, 400 MHz): δ 5.35 (1H, tq, J=7.9, 1.2 Hz, CH at C2), 3.73 (2H, d, J=7.9 Hz, CH₂ at C1), 2.82 (3H, s, —SCH3), 2.09 (2H, t, J=7.2 Hz, CH₂ at C4), 1.74 (3H, s, CH₃ at C3), 1.52 (1H, sept, J=6.6 Hz, CH at C15), 1.47-0.99 (21H, m), 0.87 (6H, d, J=6.5 Hz, CH₃ at C15), 0.85 (3H, d, J=5.4 Hz, CH₃ at C11 or C7), 0.84 (3H, d, J=6.6 Hz, CH₃ at C11 or C7). ¹³C NMR (CDCl₃, 100 MHz): δ 146.1, 109.8, 54.1, 39.4, 38.7, 38.4, 36.8, 36.7, 36.6, 36.0, 32.1, 32.0, 27.3, 24.4, 24.1, 23.8, 22.1, 22.0, 19.1, 19.0, 16.0. HRMS m/z [M+Na]⁺ Calcd for C₂₁H₄₂O₂S 381.2803; Found 381.2786.

<1-4>

### Synthesis of PT42 (Formula (1))

To a solution of phytol (CAS number: 7541-49-3) (200 mg, 0.67 mmol, 1.0 eq) in dry dichloromethane (0.68 mL) was added dimethyl sulfide (500 μL, 420 mg, 6.7 mmol, 10 equiv) at −20° C. The mixture was warmed to 0° C. over 30 min, and a diethyl ether solution of tetrafluoroborate (54 wt %, 249 UL, 296 mg, 3.37 mmol, 5.0 equiv) was then added. The reaction mixture was subsequently allowed to warm to room temperature over 4 h and stirred for an additional 20 h. Completion of the reaction was confirmed by TLC (chloroform/methanol=9:2). Ethyl acetate was added, and the mixture was washed twice with saturated aqueous sodium bicarbonate. The combined organic layer was dried over sodium sulfate, and the solvent was removed under reduced pressure. The resulting residue was suspended in hexane and transferred to a centrifuge tube. After centrifugation, the supernatant was removed to afford PT42 as a white solid (243 mg, 84%).

The NMR chemical shifts and mass spectrometric data for the product obtained as described above are presented below.

¹H NMR (CDCl₃, 400 MHz): δ 5.23 (1H, tq, J=8.1, 1.1 Hz, CH at C2), 4.05 (1H, d, J=8.1 Hz, CH₂ at C1), 2.85 (6H, s, —S⁺CH₃), 2.11 (2H, m, CH₂ at C4), 1.81 (3H, d, J=0.88 Hz, CH₃ at C3), 1.52 (1H, sept, J=6.6 Hz, CH at C15), 1.47-0.99 (18H, m), 0.87 (6H, d, J=4.6 Hz, CH₃ at C15), 0.85 (3H, d, J=6.5 Hz, CH₃ at C11 or C7), 0.85 (3H, d, J=7.5 Hz, CH₃ at C11 or C7). ¹³C NMR (CDCl₃, 100 MHz): δ 153.1, 107.6, 40.4, 40.3, 39.4, 37.4, 37.4, 37.3, 36.8, 32.8, 32.7, 28.0, 25.3, 24.8, 24.5, 22.9, 22.6, 19.7, 19.6, 17.0. ¹⁹F NMR (CDCl₃, 376 MHz): δ −149.85, −149.90. HRMS m/z [M]⁺ Calcd for C₂₂H₄₅S 341.3242; Found 341.3237.

<1-5>

### Synthesis of PT43 (Formula (2))

To a solution of phytol (CAS number: 7541-49-3) (200 mg, 0.67 mmol, 1.0 equiv) in dry dichloromethane (0.68 mL) was added diethyl sulfide (720 μL, 610 mg, 6.7 mmol, 10 equiv) at −20° C. The mixture was warmed to 0° C. over 30 min, and a diethyl ether solution of tetrafluoroborate (54 wt %, 249 UL, 296 mg, 3.37 mmol, 5.0 equiv) was then added. The reaction mixture was subsequently allowed to warm to room temperature over 4 h, and stirred for an additional 20 h. Completion of the reaction was confirmed by TLC (chloroform/methanol=9:2). Ethyl acetate was added, and the mixture was washed three times with saturated aqueous sodium bicarbonate. The combined organic layer was dried over sodium sulfate, and the solvent was removed under reduced pressure to afford PT43 as a brown oil (263 mg, 85%).

The NMR chemical shifts and mass spectrometric data for the product obtained as described above are presented below.

¹H NMR (CDCl₃, 400 MHz): δ 5.23 (1H, tq, J=8.1, 1.1 Hz, CH at C2), 4.08 (1H, d, J=8.0 Hz, CH₂ at C1), 3.37 (2H, q, J=7.5 Hz, —S⁺CH₂), 3.36 (2H, q, J=7.4 Hz, —S⁺CH₂), 2.09 (2H, m, CH₂ at C4), 1.82 (3H, d, J=0.88 Hz, CH₃ at C3), 1.52 (1H, sept, J=6.6 Hz, CH at C15), 1.52 (6H, t, J=7.5 Hz, —S⁺CH₂CH₃), 1.46-0.97 (19H, m), 0.87 (6H, d, J=6.4 Hz, CH₃ at C15), 0.85 (3H, d, J=6.1 Hz, CH₃ at C11 or C7), 0.85 (3H, d, J=6.6 Hz, CH₃ at C11 or C7). ¹³C NMR (CDCl₃, 100 MHz): δ 151.9, 108.5, 40.2, 39.4, 37.9, 37.4, 37.4, 37.3, 36.8, 33.5, 32.8, 32.7, 28.0, 25.2, 24.8, 24.5, 22.7, 22.6, 19.7, 19.6, 17.0, 9.7. ¹⁹F NMR (CDCl₃, 376 MHz): δ −149.79, −149.85. HRMS m/z [M]⁺ Calcd for C₂₄H₄₉S 369.3555; Found 369.3552.

<1-6>

### Synthesis of PT44 (formula (3))

To a solution of phytol (200 mg, 0.67 mmol, 1.0 equiv) in dry dichloromethane (0.68 mL) was added diisopropyl sulfide (970 μL, 800 mg, 6.7 mmol, 10 equiv) at −20° C. The mixture was warmed to 0° C. over 30 min, and a diethyl ether solution of tetrafluoroborate (54 wt %, 249 ML, 296 mg, 3.37 mmol, 5.0 equiv) was then added. The reaction mixture was subsequently allowed to warm to room temperature over 4 h and stirred for an additional 20 h. Completion of the reaction was confirmed by TLC (chloroform/methanol=9:2). Saturated aqueous sodium bicarbonate was added, and the reaction mixture was extracted three times with ethyl acetate. The combined organic layers were dried over sodium sulfate, and the solvent was removed under reduced pressure. The resulting residue was suspended in hexane and transferred to a centrifuge tube. After centrifugation, the supernatant was removed to afford PT44 as a light brown solid (247 mg, 76%).

The NMR chemical shifts and mass spectrometric data for the product obtained as described above are presented below.

¹H NMR (CDCl₃, 400 MHz): δ 5.24 (1H, tq, J=7.8, 1.1 Hz, CH at C2), 4.07 (1H, d, J=7.7 Hz, CH₂ at C1), 3.94 (2H, sept, J=6.8 Hz, —S⁺CH), 2.07 (2H, m, CH₂ at C4), 1.81 (3H, s, CH₃ at C3), 1.62 (6H, d, J=6.8 Hz, —S⁺CHCH₃), 1.60 (6H, d, J=6.9 Hz, —S⁺CHCH₃), 1.52 (1H, sept, J=6.6 Hz, CH at C15), 1.46-0.99 (20H, m), 0.87 (6H, d, J=6.9 Hz, CH₃ at C15), 0.85 (3H, d, J=7.2 Hz, CH₃ at C11 or C7), 0.84 (3H, d, J=6.7 Hz, CH₃ at C11 or C7). ¹³C NMR (CDCl₃, 100 MHz): δ 149.2, 110.7, 44.4, 40.1, 39.3, 37.4, 37.4, 37.3, 36.7, 34.2, 32.8, 32.6, 28.0, 25.0, 24.8, 24.4, 22.6, 19.7, 19.6, 19.3, 18.4, 16.8. ¹⁹F NMR (CDCl₃, 376 MHz): δ −150.40, −150.45. HRMS m/z [M]⁺ Calcd for C₂₆H₅₃S 397.3868; Found 397.3870.

<1-7>

### Synthesis of PT49 (Formula (6))

To a solution of phytol (200 mg, 0.67 mmol, 1.0 equiv) in dry dichloromethane (0.68 mL) was added di-tert-butyl sulfide (1.2 mL, 0.99 g, 6.7 mmol, 10 equiv) at −20° C. The mixture was warmed to 0° C. over 30 min, and a diethyl ether solution of tetrafluoroborate (54 wt %, 249 ML, 296 mg, 3.37 mmol, 5.0 equiv) was then added. The reaction mixture was subsequently allowed to warm to room temperature over 4 h, and stirred for an additional 20 h. Completion of the reaction was confirmed by TLC (chloroform/methanol=9:2). Ethyl acetate was added, and the mixture was washed twice with saturated aqueous sodium bicarbonate. The combined organic layers were dried over sodium sulfate, and the solvent was removed under reduced pressure. The resulting residue was suspended in hexane and transferred to a centrifuge tube. After centrifugation, the supernatant was removed to afford the crude collected product PT49 as a light brown solid.

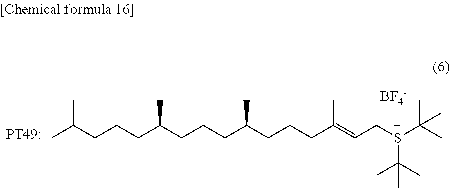

### 2. Evaluation of Growth-Inhibiting Activity of Compound on Human Leukemia T Cell Line Jurkat Cells

The cancer cell growth-inhibiting activity of each of the above samples was evaluated using the human leukemia T cell line, Jurkat.

A suspension of Jurkat cells was prepared at 2×10⁵ cells/mL, and 50 μL of the suspension was seeded into each well of a 96-well plate. The evaluation sample was dissolved in DMSO and subsequently diluted 500 fold with culture medium. Immediately after dilution, 50 μL of the resulting solution was added to each well to initiate treatment. After incubation for 72 h, 10 μL of the viable cell counting reagent SF was added to each well. Following incubation for 2 to 3 hours to allow color development, absorbance at 450 nm and 630 nm was measured using a microplate reader. Cell viability was then calculated using the following formula (F1).

$$Cell viability ( \% ) = [ ( As - Ab ) / ( Ac - Ab ) ] \times 100 \qquad ( F1 )$$

As, Ab, and Ac are defined as follows.
As: absorbance of evaluation well
Ab: absorbance of negative control well
Ac: absorbance of control well

The respective “absorbances” in the above formula were calculated by subtracting the absorbance at 630 nm (A630) from the absorbance at 450 nm (A450) and was represented by “A450-A630”. Furthermore, to calculate IC₅₀, a linear approximation was performed on a graph showing the logarithmic values of the two concentrations that most narrowly bracketed a cell viability of 50%. The results are shown in Table 1.

TABLE 1

| | [symbol] | Formula |
|---|---|---|
| Compound | IC₅₀ (μM) | number |
| 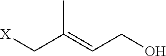 phytol | 12.8 | (C1) |
| 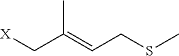 PT37 | >100 | (C2) |
| 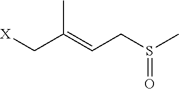 PT40 | 30 | (4) |
| 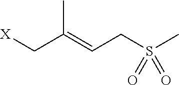 PT41 | >100 | (5) |
| 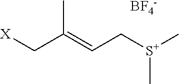 PT42 | 1.9 | (1) |
| 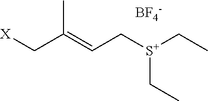 PT43 | 0.55 | (2) |
| 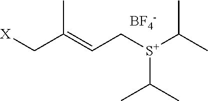 PT44 | 0.43 | (3) |

X = 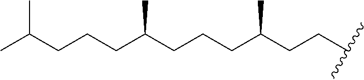 [SMILES: *CC[C@H](C)CCC[C@H](C)CCCC(C)C] [symbol]
indicates data missing or illegible when filed

Phytol (formula (C1)) and PT37 (formula (C2)) were used as comparative examples. The IC₅₀ values against Jurkat cells were as follows: phytol (formula (C1)), 12.8 μM; PT37 (formula (C2)), 100 μM or more; PT40 (formula (4)), 30 μM; and PT41 (formula (5)), 100 μM or more. The novel compound PT40 (formula (4)) also exhibited the cancer cell growth-inhibitory effect.

PT42 (formula (1)), PT43 (formula (2)), and PT44 (formula (3)) demonstrated strong inhibitory effect against Jurkat cells, with IC₅₀ values of 1.9 μM, 0.55 μM, and 0.41 μM, respectively.

### INDUSTRIAL APPLICABILITY

The cancer cell growth-inhibiting composition described herein may be suitably used for the treatment and prevention of cancer.

1. A compound having a structure represented by the formula (123): 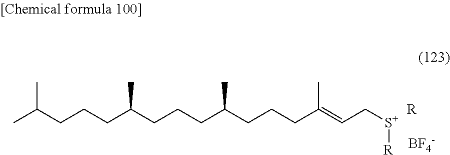
R is an alkyl group of C1 to C3.

2. A cancer cell growth-inhibiting composition comprising, as an active ingredient, at least one type of the compound of claim 1 or a pharmaceutically acceptable salt thereof.

3. A processed food comprising at least one type of the compound of claim 1 or a pharmaceutically acceptable salt thereof.

4. A compound having a structure represented by the formula (4):

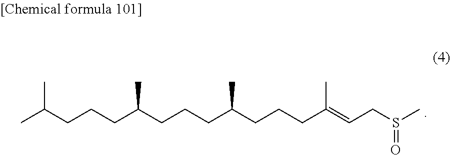 [SMILES: C/C(=C\CS(C)=O)CCC[C@H](C)CCC[C@H](C)CCCC(C)C]

5. A compound having a structure represented by the formula (5): 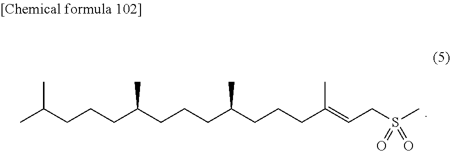 [SMILES: C/C(=C\CS(C)(=O)=O)CCC[C@H](C)CCC[C@H](C)CCCC(C)C]
